(19) United States
(12) Patent Application Publication
(10) Pub. No.: US 2025/0143677 A1
(43) Pub. Date: May 8, 2025

(54) Elastic head reflex hammer

(71) Applicant: Ben Ciechanover, Edmonton (CA)

(72) Inventor: Ben Ciechanover, Edmonton (CA)

(21) Appl. No.: 18/500,111

(22) Filed: Nov. 2, 2023

(51) Int. Cl. A61B 9/00

(52) U.S. Cl. CPC A61B 9/00

(57) ABSTRACT

A hand-held reflex hammer made up of an elastic head with a mass dense distal portion used in the assessment, diagnosis, or other functions with a person, animal, or other living thing. A method of balancing the weight of the reflex hammer and elasticity of the head with the utility of the device to elicit deep tendon reflex responses upon percussion, impulse, or force applied against a tendon or muscle. A dense material is positioned in or outside the shaft of the device, thereby freeing the head to consist of an elastic material while maintaining the normal functional characteristics of a reflex hammer such as modulating the mechanical advantage of the hammer while enabling modulation of the percussion, impulse, or force exerted onto the target area. The reflex hammer may be swung or otherwise accelerated before impacting the target, transferring the momentum of the device onto the target while minimizing risk of pain, injury, or harm towards the recipient or user of the device. This device may be used for medical, veterinary, chiropractic, naturopathic, or other purposes. The shape of the reflex hammer head may be designed in different shapes, including animals, cars, planes, plants, weapons, living things, objects, real, virtual, or imagined.

### FIELD OF THE INVENTION

The present invention provides a device, tool, or accessory used for elicitation of a reflex response through torque and or mechanical advantage along an axis before impacting another object, person, or material with a predictable impulse, percussion, impact force. Among the uses for the invention is the elicitation of deep tendon responses on neurological exams by delivering a consistent and predictable impulse, impact force, or percussion, thereby enabling more reliable assessments while limiting pain via excessively forceful impact.

### BACKGROUND

Percussion hammers, later named reflex hammers, were inspired by the practice of thumbing a wine cast to find the level of fluid within the cast. In 1875 Erb and Westphal first reported the diagnostic utility of the patellar or knee jerk reflex in examination of the nervous system, then by the 20th century there was an influx of percussion hammers with an assortment different types and shapes of the handle, shaft, and striking head of the device. While initially percussion hammers utilized more rigid heads or striking areas, later versions utilized softer or more elastic materials to limit pain or risk of injury to the recipient or handler of the hammer. A common theme in the development of later reflex hammers was the increased weight of the head, combined in some cases with a longer, sometimes flexible, handle, which was shown to increase the hammer's effectiveness. The weight of the reflex hammers was generally between 50 and 250 grams with a length generally between 10 and 40 centimetres. The material for the handle or shaft, along with the weight for the head was made of metal alloy, and the head of the hammer included a rubber piece to cushion and modulate the percussion, impulse, or force exerted by the hammer.

As of the 21^{st} century, most neurologists, neurosurgeons, physiatrists, and many other medical practitioners use reflex hammers to assess the nervous system using different grading systems. An effective reflex hammer is one which can reliably elicit a reflex response when striking a muscle or tendon in a consistent way and while minimizing pain and risk of injury to the recipient of the striking force, which often includes the patient or practitioner. However, common reflex hammers being used may be inconsistent in their delivery of the percussion, impulse, or force elicited by the hammer, may cause excessive force that may potentially leads to injury, may be too bulky, or may be too expensive. Modulating the forces applied by the hammer enables the user to provide a safer and more effective delivery of the force. In addition, freeing the head of the device to be made up of polymer by maintaining a dense weighed portion among or within the shaft of the device allows for greater flexibility in the shapes of the head and directionality of the impact.

### SUMMARY

The invention provides for a method to construct reflex hammers and or percussion hammers used in the elicitation of impulse, percussion, or impact force by utilizing the elasticity of the head of the device while maintaining the mechanical advantage of the hammer using dense material within or among the distal aspect of the hammer. The invention outlines multiple ways to modulate the elasticity of the hammer head along with the mechanical advantage of the hammer, which includes but is not limited to the length of the shaft, weight of the device, mechanical advantage along the axis of the device, and elasticity of the head of the device. Placing a higher density within or among the distal aspect of the shaft of the device increases its mechanical advantage while freeing the head of the device to be made of a lower density material such as a polymer, which further modulates the force applied while increasing safety and effectiveness of the device. Having greater flexibility in the design of the reflex hammer head allows for greater diversity in the shape of the reflex hammer, which may further be tailored towards mimicry of other shapes such as cars, planes, animals, plants, weapons, living things, objects, real, virtual, or imagined.

### DESCRIPTION OF THE FIGURES

FIG. 1 is a diagram of 4 types of reflex hammers utilizing differences in density. The white color represents low density of material or a hollow shaft. The grey color represents medium density, such as that of a polymer. The black color represents high density of material.

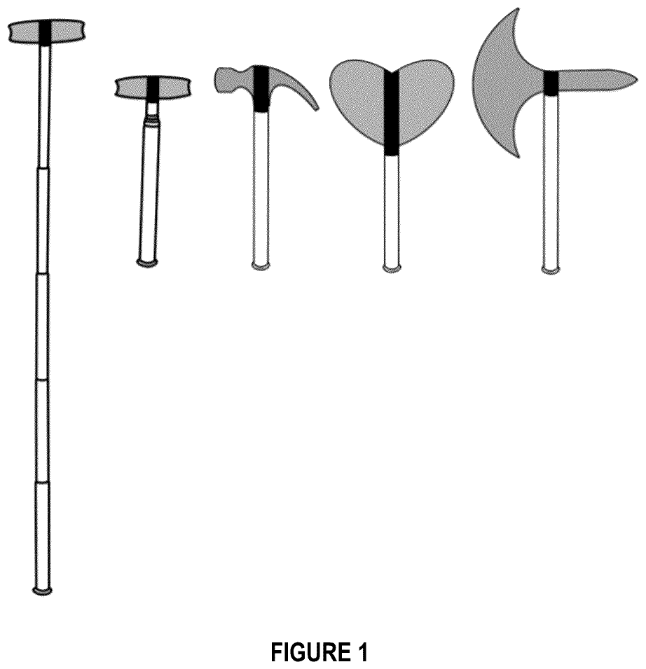

FIG. 2 is a diagram showing a reflex hammer with a denser weighted portion indicated in black, elastic polymer in grey, hollow telescopic shaft that is collapsed on the left and expanded on the right. The white color represents low density of material or a hollow shaft. The proximal end of the reflex hammer contains an elevated edge that may be used for the elicitation of deep pain responses.

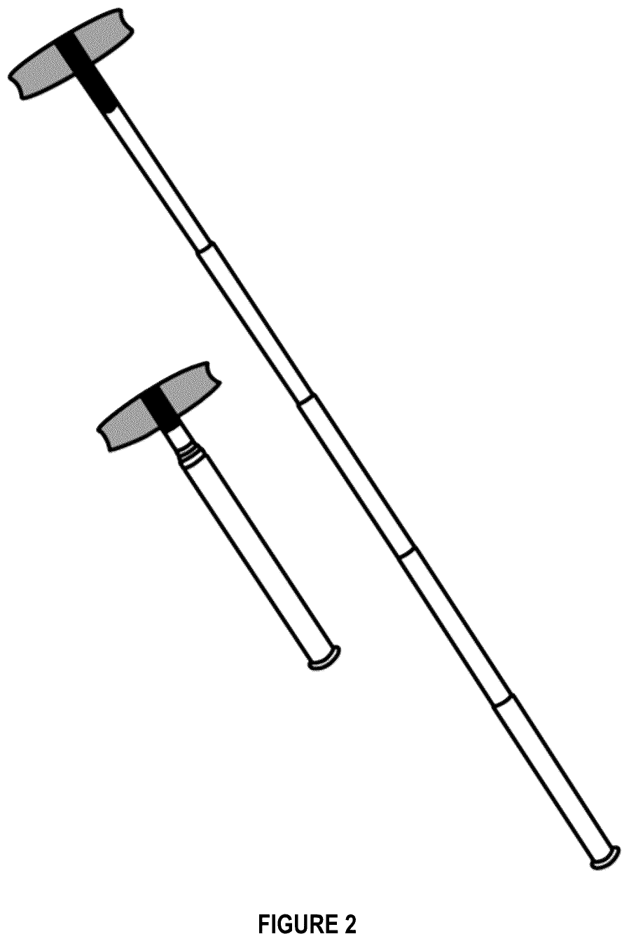

FIG. 3. Is a diagram of a reflex hammer with a denser weighted portion indicated in black, elastic polymer in grey, and low density or hollow shaft indicated in white color. Using specific parameters this example shows how a common hammer-like shape may be constructed with an elastic polymer head and used as a reflex hammer.

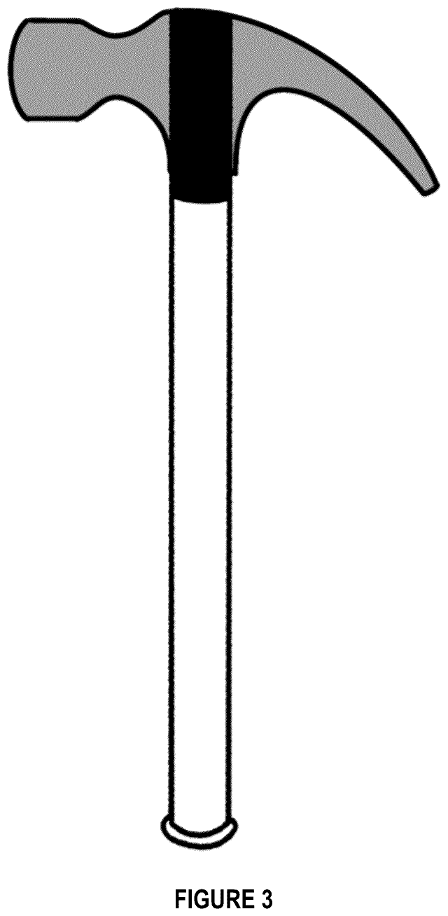

FIG. 4. Is a heart shaped reflex hammer with a denser weighted portion indicated in black, elastic polymer in grey, and low density or hollow shaft indicated in white color.

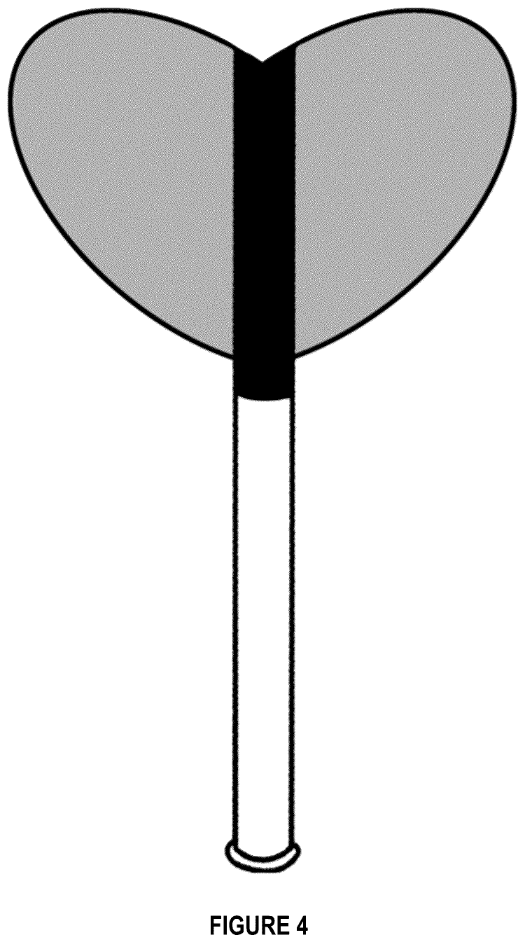

FIG. 5. Is a neurological tomahawk or axe design used as a reflex hammer with a denser weighted portion indicated in black, elastic polymer in grey, and low density or hollow shaft indicated in white color.

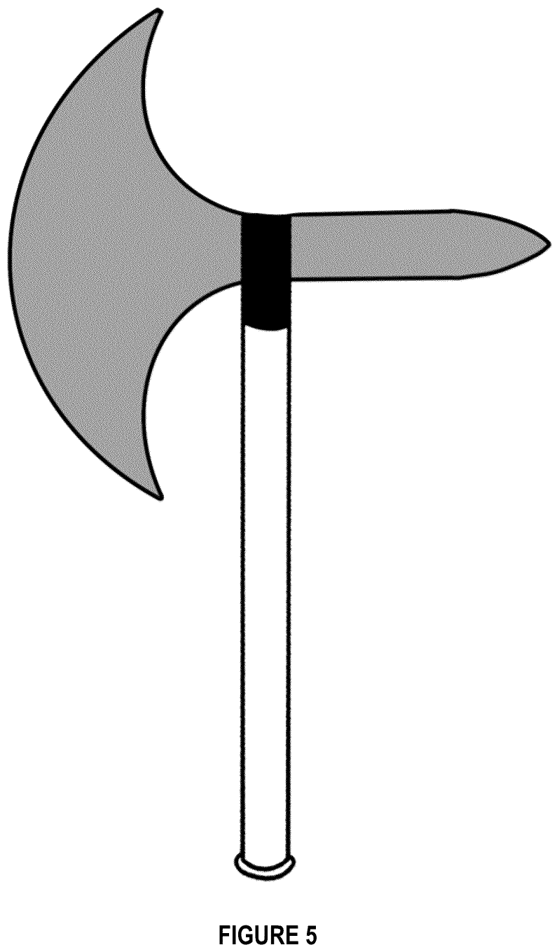

### DETAILED DESCRIPTION

The present invention provides the description and specific examples of the methods to construct optimal reflex hammers.

As used herein, a “device” refers to an object made for a particular purpose, especially when used in a mechanical form.

As used herein, a “shaft” refers to a long, narrow part or section forming the body or handle of a device or tool.

As used herein, “percussion” refers to striking of an object with or against another with some degree of force.

As used herein, “density” or “dense” refers to the degree of compactness of a substance or material, especially in the context of mass.

As used herein, “modulation” refers to the exertion of a modifying or controlling influence onto another object, person, or material.

As used herein, “mass” refers to the intrinsic property of matter involving the resistance that the body of mater offers to a change in its speed or position upon application of a force.

As used herein, “polymer” refers to a substance that has a molecular structure consisting of a large number of similar units bonded together.

As used herein, “elastic” refers to an object or material that is able to resume its normal shape spontaneously after contraction, dilation, or distortion.

As used herein, “head” refers to the distal part of an object, person, or animal, often separated from the rest of the body or object. Within the context of the device, “head” refers to any material constructed near the distal portion of the device that may be intended to strike or percuss against another item.

As used herein, “tool” refers to a device or implement, especially held in the hand yet may be used otherwise as well, used to carry out a particular function.

As used herein, “mechanical advantage” refers to the ratio of the force produced by a machine to the force applied to it.

As used herein, “hardness” refers to the quality or measurement of resistance to localized deformation, such as indentation, scratch, induced mechanically either by pressing, pressure, or abrasion established via a shore durometer.

As used herein, “Shore hardness” refers to the measurement or scale of resistance of a material to indentation.

As used herein, “30A” in the context of Shore hardness refers to the hardness of common mouse pads.

As used herein, “40A” in the context of Shore hardness refers to the hardness of common paper erasers.

As used herein, “50A” in the context of Shore hardness refers to the hardness of common pencil erasers.

As used herein, “60A” in the context of Shore hardness refers to the hardness of common tire tread.

As used herein, “70A” in the context of Shore hardness refers to the hardness of common running shoe soles.

As used herein, “80A” in the context of Shore hardness refers to the hardness of common leather belts.

### EXAMPLES

### Example 1

Reflex hammers are used is the elicitation of deep tendon reflex responses. Some of the sites traditionally used for deep tendon reflexes include the biceps tendon, brachioradialis tendon, triceps tendon, pectoralis tendon, patellar tendon, Achilles tendon, yet other reflex responses may be elicited in many other skeletal muscles as well. The deep tendon reflex is caused by the stretch action on the tendon elicited by an outside force, causing a muscle response by the contraction of the muscle triggered by the tendon stretch. However, delivering a consistent impulse against the tendon of a patient is sometimes not sufficient for a reliable tendon response as patients have a diversity of tendon lengths, fatty composition above and below the tendon, along with other individual differences. By enabling modulation of the impact force and limiting excessively high impulses to the patient's tendons we reduce pain and risk of injury to patients.

The invention claimed is:

1. A device, tool, instrument, or forms of constructs used to elicit reflex responses using an elastic head with a heavier or dense material elsewhere in the device near the distal portion of the device.

2. The device of claim 1, wherein the elastic head is made of polymer, organic, or inorganic substance.

3. The device of claim 1, wherein the distal aspect of the device consists of higher density material within or made up of the shaft.

4. The device of claim 1, wherein the shaft of the device may be telescopic, expandable, or of constant length.

5. The device of claim 1, wherein the proximal aspect of the device may contain, house, feature, or otherwise display another device, tool, or other accessory not otherwise specified.

6. The device of claim 1, wherein the shaft may be made up of polymer, metal, organic, or inorganic substance.

7. The device of claim 1, wherein the higher density material is made up of a polymer, metal, organic, or inorganic substance or substances.

8. The device of claim 1, wherein the device may be hand-held, while a proximal end of the device is affixed manually, mechanically, or automatically.

9. The device of claim 1, wherein the shaft of the device may be of proportional length to the application in use.

10. The device of claim 1, wherein the mass of the device may be adjusted per the application in use.

11. A reflex hammer constructed of mostly an elastic head.

12. The reflex hammer of claim 11, wherein the elastic head is made of polymer, organic, or inorganic substance.

13. The reflex hammer of claim 11, wherein the distal aspect of the device consists of higher density material within or made up of the shaft.

14. The reflex hammer of claim 11, wherein the head of the reflex hammer is made up of an elastic material with shore hardness between 30A and 80A, yet preferably between 40A and 70A, inclusive, and include 50A, 60A, and all other shore hardness value between 30A and 80A.

15. The reflex hammer of claim 11, wherein weight distribution of the reflex hammer is greater at the distal end compared to the proximal end where the reflex hammer is held.

16. The reflex hammer of claim 11, wherein the handle or shaft is made up of a rigid or flexible material.
